(19) United States
(12) Patent Application Publication
(10) Pub. No.: US 2024/0278144 A1
(43) Pub. Date: Aug. 22, 2024

(54) DEHYDRATION SYSTEM WITH SPLIT PERMEATE OR RETENTATE HEADER

(71) Applicant: Whitefox Technologies Limited, London (GB)

(72) Inventors: Virginia ANDRADE, Calgary (CA); Jin Ming ZHOU, Calgary (CA); Stephan BLUM, Calgary (CA); Thiago RIGHI, Calgary (CA)

(73) Assignee: Whitefox Technologies Limited, London (GB)

(21) Appl. No.: 18/581,636

(22) Filed: Feb. 20, 2024

Related U.S. Application Data
(60) Provisional application No. 63/447,444, filed on Feb. 22, 2023.

(51) Int. Cl. B01D 3/14; B01D 5/00; C07C 29/80

(52) U.S. Cl. CPC B01D 3/145; B01D 3/148; B01D 5/006; C07C 29/80

(57) ABSTRACT

An organic solvent production system and method are provided in which permeate and/or retentate streams are collected from a set of membranes or other purification devices via one or more headers to direct the streams to various downstream systems for further processing and/or heat recovery against colder streams.

### CROSS-REFERENCE TO RELATED APPLICATIONS

The present disclosure claims the benefit of U.S. Provisional Patent Application No. 63/447,444 entitled “DEHYDRATION SYSTEM WITH SPLIT PERMEATE OR RETENTATE HEADER” and filed on 2023 Feb. 22, which is incorporated herein by reference in its entirety BACKGROUND

To refine various solvents, various adulterants are removed from the base to result in a purified solvent. For example, when refining an organic solvent (e.g., ethanol, isobutanol, isopropanol) produced via fermentation, adulterants such as water and solids from fermentation process are removed. Depending on desired specifications, typical refining processes use a series of distillation steps combined with a dehydration unit operation to achieve 99 vol. % or higher solvent content. For example, a solvent production system is typically designed to achieve a final product having a particular solvent purity based on the desired application of the final product. For example, the typical organic solvent production system may be designed to achieve an organic solvent purity suitable for fuel applications (e.g., 99 vol. % for fuel grade in the United States or 99.7 wt % for fuel grade in the European Union) or may be designed to achieve a solvent purity suitable for pharmaceutical applications (e.g., 99.95 wt %). The typical organic solvent production system, however, is designed to achieve only a single final product of the same purity.

### SUMMARY

The present disclosure provides new and innovative systems and methods for solvent production that provide a wider array of opportunities for permeate heat integration than typical systems and methods by reducing the duty on an individual permeate condenser. With the decreased duty on each permeate condenser, it becomes easier to integrate the permeate energy with other streams in the provided solvent production system. For instance, if the total generated permeate flow is directed to a single permeate condenser (as in typical systems), then the size of the single permeate condenser required to condense the permeate at vacuum pressures becomes impractical. Stated differently, the single permeate condenser would need an impractically large heat transfer area in order to transfer heat from the permeate stream to a cold stream against which the latent heat from the permeate stream is desired to be recovered. As such, the duty on the single permeate condenser would be too large to integrate the permeate energy with other suitable streams.

In some aspects, the provided system splits the total generated permeate flow into more than one stream in order to decrease the duty on each permeate condenser to which a respective stream of the more than one streams is directed. In one aspect, the provided system may include at least one permeate header to which permeate vapor from some membranes, but not all membranes, of the provided system is directed. The permeate header directs the permeate vapor to a first permeate condenser, and the permeate not directed to the permeate header is directed to a second, different permeate condenser (e.g., via a second permeate header). In another aspect, the permeate vapor from all of the membranes of the provided system may be directed to a single permeate header that then splits off to direct a first portion of the permeate vapor to a first permeate condenser and a second portion of the permeate vapor to a second, different permeate condenser. The amount of permeate vapor directed to each permeate condenser may be varied based on process control. As such, in either aspect, the duty of each individual permeate condenser is decreased as compared to a permeate condenser that receives all of the permeate. With the decreased duty, a permeate condenser of a practical size may be used, which enables recovering the permeate vapor latent heat against a suitable cold stream.

In some aspects, the provided system splits the total generated retentate flow into more than one stream in order to increase the flexibility of the organic solvent purity of the final product produced by the provided system. For example, one stream of retentate may result in a final product having a higher organic solvent purity than a second stream of retentate. As such, the provided system may provide final product for more than one application (e.g., fuel grade and pharmaceutical grade).

In an example, a system is provided that includes a distillation column configured to receive a feed stream and generate a distillation column overhead stream from the feed stream; a stripper column arranged to indirectly receive a stripper feed stream including at least a portion of the distillation column overhead stream, the stripper column configured to generate a stripper column overhead stream from the stripper feed stream; at least three membranes arranged to each receive a respective portion of a membrane feed stream including at least a portion of the stripper column overhead stream, wherein each of the at least three membranes is configured to generate from its respective portion of the membrane feed stream a respective permeate and a respective retentate, the respective permeates together composing a total permeate and the respective retentates together composing a total retentate; a first condenser; and a second condenser, wherein each of the at least three membranes is in fluid communication with at least one of the first condenser and the second condenser such that a first non-zero portion of the total permeate generated by the at least three membranes is directed to the first condenser and a second non-zero portion of the total permeate generated by the at least three membranes is directed to the second condenser.

In some aspects, a first membrane and a second membrane of the at least three membranes are each in fluid communication with a first permeate header such that the respective permeates of the first and second membranes are both directed to the first permeate header, and wherein the first permeate header is in fluid communication with the first condenser and not with the second condenser such that the first non-zero portion of the total permeate includes the respective permeates generated by the first and second membranes.

In some aspects, a third membrane of the at least three membranes is in fluid communication with the second condenser and not with the first condenser such that the second non-zero portion of the total permeate includes the permeate generated by the third membrane.

In some aspects, the at least three membranes includes a third membrane and a fourth membrane, wherein the third and fourth membranes are each in fluid communication with a second permeate header such that the respective permeates of the third and fourth membranes are both directed to the second permeate header, and wherein the second permeate header is in fluid communication with the second condenser and not with the first condenser such that the second non-zero portion of the total permeate includes the respective permeates generated by the third and fourth membranes.

In some aspects, the system is further comprising a first valve and a second valve, the first valve configured and arranged to adjust a flow of the first non-zero portion of the total permeate, and the second valve configured and arranged to adjust a flow of the second non-zero portion of the total permeate.

In some aspects, the first and second valves are arranged such that adjustment of at least one of the first valve and the second valve adjusts an amount of the first portion of the total permeate as compared to the second portion.

In some aspects, the system is further comprising a first process stream directed to the first condenser, wherein the first non-zero portion of the total permeate exchanges energy with the first process stream in the first condenser.

In some aspects, the system is further comprising a second process stream directed to the second condenser, wherein the second non-zero portion of the total permeate exchanges energy with the second process stream in the second condenser.

In some aspects, the organic solvent is one in the group consisting of ethanol, iso-butanol, and isopropanol.

In some aspects, the organic solvent is ethanol.

In some aspects, each of the at least three membranes is in fluid communication with an evaporation system, the evaporation system including a plurality of evaporators, such that the respective retentates generated by the at least three membranes are directed to at least one evaporator of the plurality of evaporators.

In some aspects, the system is further comprising a stripper/rectifier column arranged to receive the distillation column overhead stream as a vapor, the stripper/rectifier column configured to generate a stripper/rectifier column overhead stream from the vaporous distillation column overhead stream, wherein the stripper column is arranged to receive at least a portion of the stripper/rectifier column overhead stream, and wherein the stripper/rectifier column overhead stream includes the at least a portion of the distillation column overhead stream.

In some aspects, the system is further comprising a molecular sieve unit arranged to receive a molecular sieve unit feed stream including at least a portion of the distillation column overhead stream, the molecular sieve unit configured to generate a regenerate stream from the molecular sieve unit feed stream, wherein the stripper column is arranged to directly receive the regenerate stream, and wherein the regenerate stream includes the at least a portion of the distillation column overhead stream.

In some aspects, each of the at least three membranes is in fluid communication with at least one of a third condenser and a fourth condenser such that a first non-zero portion of the total retentate generated by the at least three membranes is directed to the third condenser and a second non-zero portion of the total retentate generated by the at least three membranes is directed to the fourth condenser.

In some aspects, the system is further comprising: a third condenser; and an evaporator, wherein each of the at least three membranes is in fluid communication with at least one of the third condenser and the evaporator such that a first non-zero portion of the total retentate generated by the at least three membranes is directed to the third condenser and a second non-zero portion of the total retentate generated by the at least three membranes is directed to the evaporator.

In an example, a method is provided that includes receiving a feed stream at a distillation column; generating a distillation column overhead stream from the feed stream via the distillation column; receiving a stripper feed stream at a stripper column, the stripper feed stream including at least a portion of the distillation column overhead stream; generating a stripper column overhead stream from the stripper feed stream via the stripper column; receiving a respective portion of a membrane feed stream at each of at least three membranes, the membrane feed stream including at least a portion of the stripper column overhead stream; generating a respective permeate and a respective retentate from the respective portion of the membrane feed stream at each respective membrane of the at least three membranes, the respective permeates together composing a total permeate and the respective retentates together composing a total retentate; directing a first non-zero portion of the total permeate to a first condenser; and directing a second non-zero portion of the total permeate to a second condenser.

In some aspects, the method further includes exchanging energy of the first non-zero portion of the total permeate with a first process stream in the first condenser.

In some aspects, the first process stream is one of the streams in the group consisting of: a 190P stream, a scrubber bottoms stream, a cook water stream, a regen stream.

In some aspects, the method further includes exchanging energy of the second non-zero portion of the total permeate with a second process stream in the second condenser.

In some aspects, the first non-zero portion of the total permeate includes all of the respective permeates generated by each of a first membrane and a second membrane of the at least three membranes, and wherein the second non-zero portion of the total permeate includes all of the permeate generated by a third membrane of the at least three membranes.

In some aspects, the second non-zero portion of the total permeate includes all of the respective permeates generated by each of the third membrane and a fourth membrane of the at least three membranes.

In some aspects, the method further includes directing a first non-zero portion of the total retentate to a third condenser; and directing a second non-zero portion of the total retentate to an evaporator.

In an example, a system is provided that includes a distillation column configured to receive a feed stream and generate a distillation column overhead stream from the feed stream; a stripper column arranged to indirectly receive a stripper feed stream including at least a portion of the distillation column overhead stream, the stripper column configured to generate a stripper column overhead stream from the stripper feed stream; at least three membranes arranged to each receive a respective portion of a membrane feed stream including at least a portion of the stripper column overhead stream, wherein each of the at least three membranes is configured to generate from its respective portion of the membrane feed stream a respective permeate and a respective retentate, the respective permeates together composing a total permeate and the respective retentates together composing a total retentate; a condenser; and an evaporator, wherein each of the at least three membranes is in fluid communication with at least one of the first condenser and the evaporator such that a first non-zero portion of the total retentate generated by the at least three membranes is directed to the condenser and a second non-zero portion of the total retentate generated by the at least three membranes is directed to the evaporator.

In some aspects, the system is further comprising: a second condenser; and a third condenser, wherein each of the at least three membranes is in fluid communication with at least one of the second condenser and the third condenser such that a first non-zero portion of the total permeate generated by the at least three membranes is directed to the second condenser and a second non-zero portion of the total permeate generated by the at least three membranes is directed to the third condenser.

In an example, a method is provided that includes receiving a feed stream at a distillation column; generating a distillation column overhead stream from the feed stream via the distillation column; receiving a stripper feed stream at a stripper column, the stripper feed stream including at least a portion of the distillation column overhead stream; generating a stripper column overhead stream from the stripper feed stream via the stripper column; receiving a respective portion of a membrane feed stream at each of at least three membranes, the membrane feed stream including at least a portion of the stripper column overhead stream; generating a respective permeate and a respective retentate from the respective portion of the membrane feed stream at each respective membrane of the at least three membranes, the respective permeates together composing a total permeate and the respective retentates together composing a total retentate; directing a first non-zero portion of the total retentate to a condenser; and directing a second non-zero portion of the total retentate to an evaporator.

In some aspects, the method further includes directing a first non-zero portion of the total permeate to a second condenser; and directing a second non-zero portion of the total permeate to a third condenser.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates example organic solvent production systems, according to aspects of the present disclosure.

FIG. 2 illustrates an example layout in which the membranes provide permeate stream outputs to multiple permeate headers directed to corresponding different downstream processes, while providing the retentate stream output to a single retentate header provided to a single downstream process, according to aspects of the present disclosure.

FIG. 3 illustrates an example layout in which the membranes provide permeate stream outputs to a single permeate header, which is later split to different downstream process, while providing the retentate stream output to a single retentate header provided to a single downstream process, according to aspects of the present disclosure.

FIG. 4 illustrates an example layout in which the membranes provide permeate stream outputs to a single permeate header provided to a single downstream process, while providing the retentate stream output to a single retentate header provided to multiple downstream process, according to aspects of the present disclosure.

FIG. 5 illustrates an example layout in which the membranes provide permeate stream outputs to a single permeate header provided to a single downstream process, while providing the retentate stream output to multiple retentate headers provided to different downstream process, according to aspects of the present disclosure.

FIG. 6 illustrates an example layout in which the membranes provide permeate stream outputs to a single permeate header provided to multiple downstream process, while providing the retentate stream output to a single retentate headers provided to multiple downstream process, according to aspects of the present disclosure.

FIG. 7 illustrates an example layout in which the membranes provide permeate stream outputs to multiple permeate headers provided to different downstream process, while providing the retentate stream output to multiple retentate headers provided to different downstream process, according to aspects of the present disclosure.

FIG. 8 illustrates an example organic solvent production system including split retentate headers, according to an aspect of the present disclosure.

FIG. 9 illustrates an example organic solvent production system including split retentate and permeate headers, according to an aspect of the present disclosure.

FIG. 10 illustrates an example organic solvent production system including a vaporizer, according to an aspect of the present disclosure.

FIG. 11 is a flowchart of an example method for operating a dehydration system with a split header design, according to an aspect of the present disclosure.

### DETAILED DESCRIPTION

The present disclosure provides new and innovative systems and methods for solvent production that provide a wider array of opportunities for permeate heat integration than typical systems and methods by reducing the duty on an individual permeate condenser. With the decreased duty on each permeate condenser, it becomes easier to integrate the permeate energy with other streams in the provided solvent production system. For instance, if the total generated permeate flow is directed to a single permeate condenser (as in typical systems), then the size of the single permeate condenser required to condense the permeate at vacuum pressures becomes impractical. Stated differently, the single permeate condenser would need an impractically large heat transfer area in order to transfer heat from the permeate stream to a cold stream against which the latent heat from the permeate stream is desired to be recovered. As such, the duty on the single permeate condenser would be too large to integrate the permeate energy with other suitable streams.

In some aspects, the provided system splits the total generated permeate flow into more than one stream in order to decrease the duty on each permeate condenser to which a respective stream of the more than one streams is directed. In one aspect, the provided system may include at least one permeate header to which permeate vapor from some membranes, but not all membranes, of the provided system is directed. The permeate header directs the permeate vapor to a first permeate condenser, and the permeate not directed to the permeate header is directed to a second, different permeate condenser (e.g., via a second permeate header). In another aspect, the permeate vapor from all of the membranes of the provided system may be directed to a single permeate header that then splits off to direct a first portion of the permeate vapor to a first permeate condenser and a second portion of the permeate vapor to a second, different permeate condenser. The amount of permeate vapor directed to each permeate condenser may be varied based on process control. As such, in either aspect, the duty of each individual permeate condenser is decreased as compared to a permeate condenser that receives all of the permeate. With the decreased duty, a permeate condenser of a practical size may be used, which enables recovering the permeate vapor latent heat against a suitable cold stream.

In some aspects, the provided system splits the total generated retentate flow into more than one stream in order to increase the flexibility of the organic solvent purity of the final product produced by the provided system. For example, one stream of retentate may result in a final product having a higher organic solvent purity than a second stream of retentate. As such, the provided system may provide final product for more than one application (e.g., fuel grade and pharmaceutical grade). Other benefits of the designs described herein will be apparent on detailed review of the provided disclosure.

One way to help increase the energy efficiency (e.g., low-pressure steam consumption) of a solvent production system is through heat integration. By using the heat (e.g., energy) of some process streams to heat other process streams, steam consumption (and therefore energy consumption) can be reduced. One process stream from which it is typically difficult to recover heat (e.g., exchange heat with another process stream), however, is permeate vapor. Various components of the presently disclosed systems may be in fluid communication with one another, such as through piping. Two components in fluid communication with one another may be in direct fluid communication (e.g., piping directly connects the two components) or may be in indirect fluid communication such that there are intermediate components or processing between the two components, such as filters, pumps, heaters, odor removal vessels, etc.

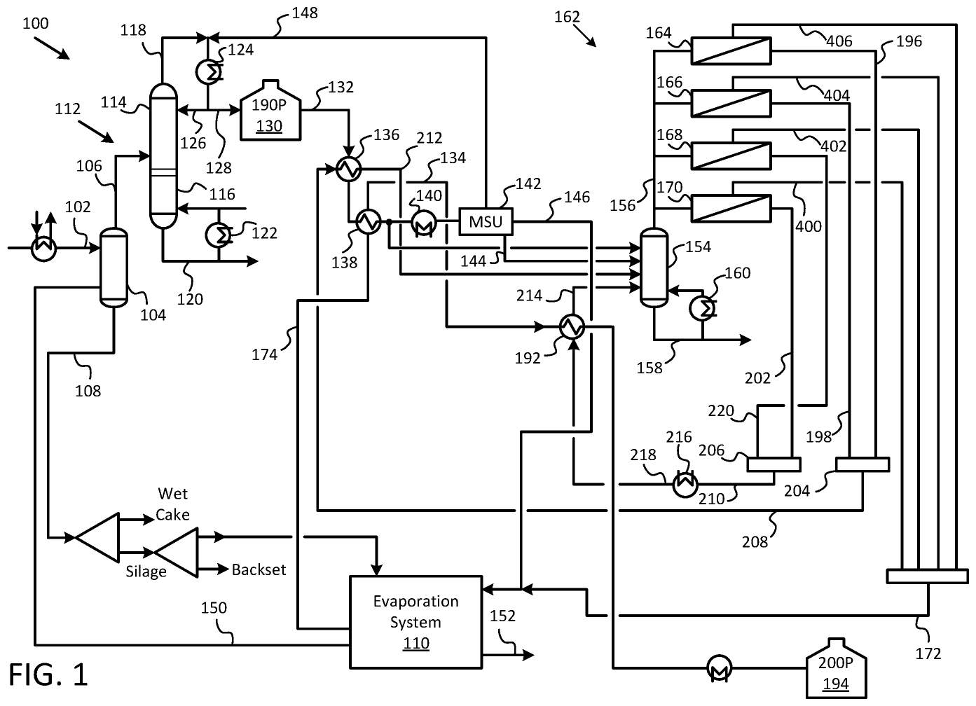

FIG. 1 illustrates an example organic solvent production system 100, according to aspects of the present disclosure. In at least some aspects, the example organic solvent production system 100 can be described as including four subsystems: (i) a feed stripping subsystem, (ii) a rectifying distillation subsystem, (iii) a dehydration subsystem, and (iv) an evaporation subsystem. Although provided with an example organic solvent production system 100, the present disclosure contemplates that various other solvent refining systems may be benefitted from the teachings provided herein, and the organic solvent production system 100 is provided as a non-limiting example.

In the feed stripping subsystem, a feed stream 102 may be directed to a distillation column 104. The feed stream 102 may include a mixture of an organic solvent, water, and solids. The distillation column 104 is structured to form a solid-freed vaporous overhead stream 106 (with a higher concentration of the organic solvent and a lower concentration of the solids than the feed stream 102) and an organic solvent-freed bottoms stream 108 (with a lower concentration of the organic solvent and a higher concentration of the solids than the feed stream 102) from the feed stream 102. In some aspects, the distillation column 104 may be driven by process vapors through direct injection, such as a vapor stream 150 from an evaporation system 110 in the evaporation subsystem. In some aspects, the distillation column 104 may be driven by vapors from process streams generated in flash vessels. In some aspects, the distillation column 104 may be driven by cook flash vapors. In some aspects, the distillation column 104 may additionally or alternatively be driven by a distillation column reboiler (e.g., with a combination of either evaporator vapors, cook flash, flash vapors generated from flashing a portion of other process streams).

In some aspects, the vaporous overhead stream 106 of the distillation column 104 may be directed straight/directly to a rectifier/stripper column 112 of the rectifying distillation subsystem. Stated differently, in such aspects, the vaporous overhead stream 106 of the distillation column 104 may be introduced into the rectifier/stripper column 112 as a vapor without first being condensed or otherwise processed. In other aspects, the vaporous overhead stream 106 may be condensed prior to being introduced into the rectifier/stripper column 112. The bottom stream 108 of the distillation column 104 may be directed towards the evaporation system 110 (e.g., to a centrifuge system and one or more evaporators as described below).

In at least some aspects, the rectifying distillation subsystem includes the rectifier/stripper column 112. In this example, the rectifier/stripper column 112 includes a rectifier 114 and a side stripper 116 integrated as a single unit. In other examples, the rectifying distillation subsystem may include a rectifier 114 in fluid communication with a separate side stripper 116. In other examples still, the rectifying distillation subsystem may include only a rectifier 114 and does not include a side stripper 116. As stated above, the distillation column 104 vaporous overhead stream 106 may be directed (e.g., straight) to the rectifier/stripper column 112. The rectifier/stripper column 112 may receive a feed stream that includes the vaporous overhead stream 106. The rectifier/stripper column 112 may form an organic solvent-rich overhead stream 118 and a bottom stream 120 from the feed stream. In various aspects, the organic solvent-rich overhead stream 118 formed by the rectifier/stripper column 112 may be between 180-proof and 190-proof. In one example, the organic solvent-rich overhead stream 118 formed by the rectifier/stripper column 112 may be 190-proof (e.g., 190P, or 95% organic solvent by volume). The organic solvent-rich overhead stream 118 may be condensed (e.g., via the condenser 124). In some aspects, a portion of the condensed organic solvent-rich overhead stream 118 may be stored in a storage tank 130 via the stream 128. In some aspects, a portion of the condensed organic solvent-rich overhead stream 118 may return to the rectifying subsystem as reflux via the stream 126. At least a portion of the condensed organic solvent-rich overhead stream 118 may be directed (e.g., from the tank 130 via the stream 132) to a separation system of the dehydration subsystem. In various aspects, the organic solvent-free bottom stream 120 may be directed to another area of the organic solvent production plant (e.g., the cook section) in which the provided organic solvent production system 100 is located. In some aspects, the side stripper 116 of the rectifier/stripper column 112 may be driven by direct vapor injection and/or steam. In other aspects, the side stripper 116 may be driven by process vapors or steam via a reboiler 122.

In at least some aspects, the dehydration subsystem includes a separation system. In this example, the separation system may include a vaporizer 140 and a set of molecular sieve beds 142 as well as at least one distillation column 154 (e.g., a stripper column) and a set of membranes 162. In other examples, the separation system may include only the at least one distillation column 154 and the set of membranes 162, and not the vaporizer 140 and the set of molecular sieve beds 142. The set of molecular sieve beds 142 is structured to generate a product stream 146 and two regenerate streams. The two regenerate streams are a regen stream 144 and a depressure stream 148. The product stream 146 is an organic solvent-rich stream (e.g., 200-proof, or 99+ vol. %). In at least some aspects, the product stream 146 may be directed to at least one of the evaporators in the evaporation system 110 of the evaporation subsystem. For example, the product stream 146 may be directed into the retentate vapor stream carried by a retentate header 172, which is directed to at least one evaporator in the evaporation system 110.

In some aspects, the product stream may be directed to a condenser in which heat is recovered against another suitable stream. For example, a product condenser can be used to recover heat from the retentate vapor against another process stream, which may be received from the evaporation system 110 or another portion of the system 100. The condensed organic solvent-rich overhead stream 118 (e.g., 190P) from the rectifier/stripper column 112 (e.g., via the stream 132 from the storage tank 130) may be directed to the vaporizer 140 which generates a vaporized stream that is directed to contact the set of molecular sieve beds 142. In some aspects, the vaporizer 140 may be driven by steam. In some examples, steam condensate from the vaporizer 140 may be flashed in a flash vessel.

In various aspects, the set of molecular sieve beds 142 may include multiple (e.g., two, three, four, etc.) beds filled with zeolite pellets, which adsorb water to produce anhydrous vapor until the zeolite pellets are saturated with water. A saturated zeolite pellet bed may be regenerated. In some instances, freshly dehydrated organic solvent may be directed to contact a saturated zeolite pellet bed to remove water from the saturated zeolite pellet bed, which produces a regenerate stream. In other instances, the regeneration is done by vacuum, generating two regenerate streams (e.g., the regen stream 144 and the depressure stream 148). The regen stream 144 may have an organic solvent concentration between 45-80 vol % and therefore must be recycled to upstream distillation for reprocessing. For example, the regen stream 144 may be directed to the distillation column 154 of the separation system. The depressure stream 148 may also be recycled to upstream distillation for reprocessing. For example, the depressure stream 148 may be directed to the rectifier 114 of the rectifier/stripper column 112 and/or the storage tank 130. In instances in which the set of molecular sieve beds 142 includes multiple zeolite pellet beds, a saturated zeolite pellet bed may be regenerated while an unsaturated zeolite pellet bed is used to dehydrate a vaporized feed stream.

In the illustrated example, the distillation column 154 generates an overhead vapor stream 156 from an organic solvent-water concentrated feed stream. In at least some aspects, the organic solvent-water concentrated feed stream may include at least a portion of the condensed organic solvent-rich overhead stream 118 generated by the rectifier/stripper column 112 (e.g., via the stream 132 from the tank 130). In some aspects, the organic solvent-water concentrated feed stream may include the regen stream 144 generated by the set of molecular sieve beds 142. In some aspects, the organic solvent-water concentrated feed stream may include permeate generated by the set of membranes 162.

In at least some aspects, the overhead vapor stream 156 is split as illustrated such that each of the membranes 164, 166, 168, and 170 of the set of membranes 162 is contacted by a portion of the overhead vapor stream 156. In some examples, the overhead vapor stream 156 generated by the distillation column 154 may be heated via steam in a superheater prior to being introduced into the membranes 164, 166, 168, and 170. In such examples, steam condensate from the superheater may be flashed in a flash vessel. The distillation column 154 may also generate a bottom stream 158 that may be directed to another area of the production plant in which the provided organic solvent production system 100 is located. In some aspects, the distillation column 154 may be driven by a reboiler 160, which may be driven by steam.

Each of the membranes 164, 166, 168, and 170 generates a permeate vapor and a retentate vapor from being contacted by a respective portion of the overhead vapor stream 156. In various aspects, each of the membranes 164, 166, 168, and 170 may be a semi-permeable membrane. While the set of membranes 162 is illustrated as having four membranes, in other examples, the set of membranes 162 may have another suitable quantity of membranes (e.g., 3, 5, 10, etc.). In some examples, the generated permeate vapor of the membranes 164, 166, 168, and 170 may be directed to the distillation column 154.

In some examples, the generated retentate vapor of each of the membranes 164, 166, 168, and 170 may be directed to at least one evaporator in the evaporation system 110 via the retentate header 172. In such examples, the retentate vapor is condensed in the at least one evaporator.

Retentate liquid energy may be further recovered against other process streams (e.g., a rectifier overhead stream, permeate liquid, scrubber bottoms, etc.). For example, the retentate liquid is illustrated in FIG. 1 heating the rectifier overhead stream (e.g., the stream 132) in an economizer 138. In at least some aspects, the cooled retentate liquid stream 134 may be directed to a tank 194 for storage.

In various aspects, the evaporation subsystem includes an evaporation system 110 including one or more evaporators. One having skill in the pertinent art will appreciate the various arrangements of evaporators in an evaporation subsystem of an organic solvent production plant. For example, in some aspects, vapors generated from a first effect evaporator may be used to drive a second effect evaporator. In some aspects, vapors generated from the second effect evaporator may be used to drive a third effect evaporator. In various aspects, the number of evaporation steps vary from two to eight (e.g., fourth effect evaporator, fifth effect evaporator, etc.). In various aspects, effect vapors from evaporators may be used to drive the distillation in the distillation subsystem. For example, the vapor stream 150 (e.g., fourth effect vapor from a fourth effect evaporator) may be used to drive the distillation column 104. In at least some aspects, the evaporation system 110 may generate a syrup 152.

In the evaporation subsystem, though not illustrated, the bottom stream 108 of the distillation column 104 may be subjected to a centrifuge system in which a concentrated solids (wet cake) and a low-solids concentration solution (thin stillage) are produced. The thin stillage may then be split into two streams: backset and evaporator feed. The evaporator feed is subjected to the evaporator system 110 to increase its solids concentrations. In at least some aspects, retentate vapor in the retentate header 172 from the separation system in the dehydration subsystem may be used to drive the evaporation system 110. In some instances, the vaporous overhead stream from a distillation column may be used to drive the evaporation.

As described above, the organic solvent production system 100 increases the potential opportunities for permeate vapor heat integration by reducing the duty placed on an individual permeate condenser.

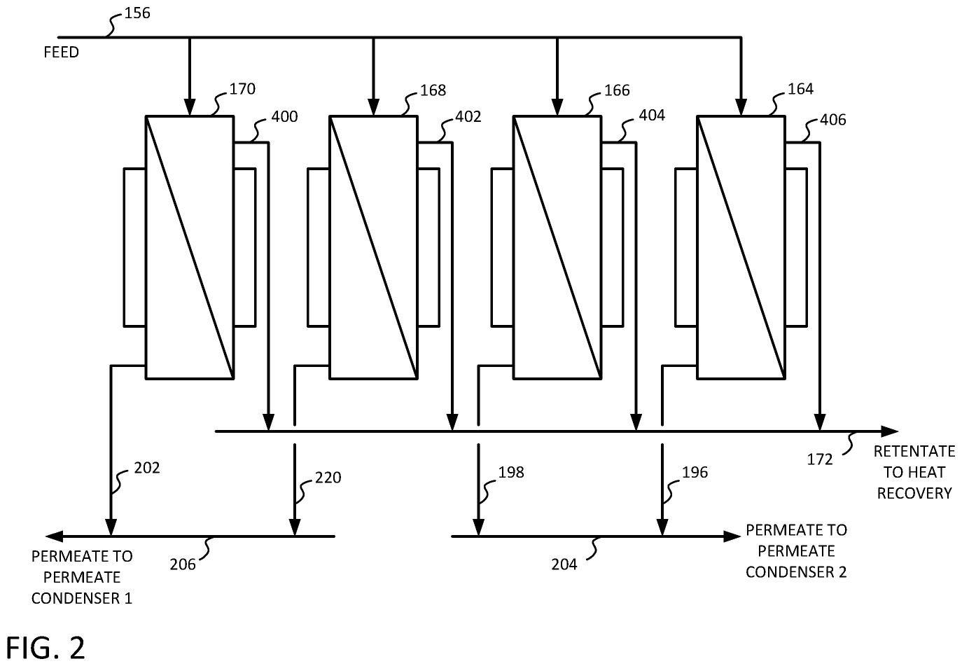

FIGS. 2-7 illustrate various layouts for the collection and splitting of the various retentate and permeate streams from the membranes 164, 166, 168, and 170. Each of FIGS. 2-7 illustrate four membranes 164, 166, 168, 170 that receive a feed stream 156 and produce corresponding permeate streams 196, 198, 220, 202 and retentate streams 406, 404, 402, 400 which may be split or combined via various headers to direct the streams for various downstream processes, which may include heat recovery. Each of the layouts may be used in various solvent production plants, such as those examples shown in FIGS. 1 and 8-10, and are given as non-limiting examples. Accordingly, the present disclosure contemplates that more than or less than four membranes may be used in various plants, and that the number of headers may be different from what is shown in FIGS. 2-7.

FIG. 2 illustrates an example layout in which the membranes provide permeate stream outputs to multiple permeate headers directed to corresponding different downstream processes (e.g., first and second permeate condensers), while providing the retentate stream output to a single retentate header provided to a single downstream process (e.g., to retentate heat recovery).

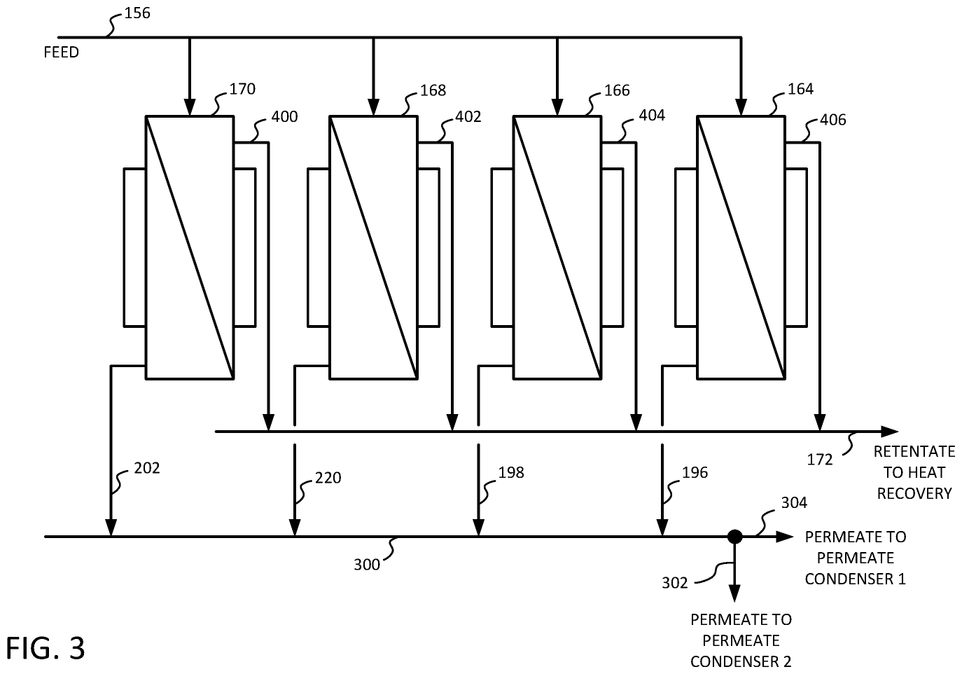

FIG. 3 illustrates an example layout in which the membranes provide permeate stream outputs to a single permeate header 300 which is later split to different downstream process (e.g., first and second permeate condensers), while providing the retentate stream output to a single retentate header provided to a single downstream process (e.g., to retentate heat recovery).

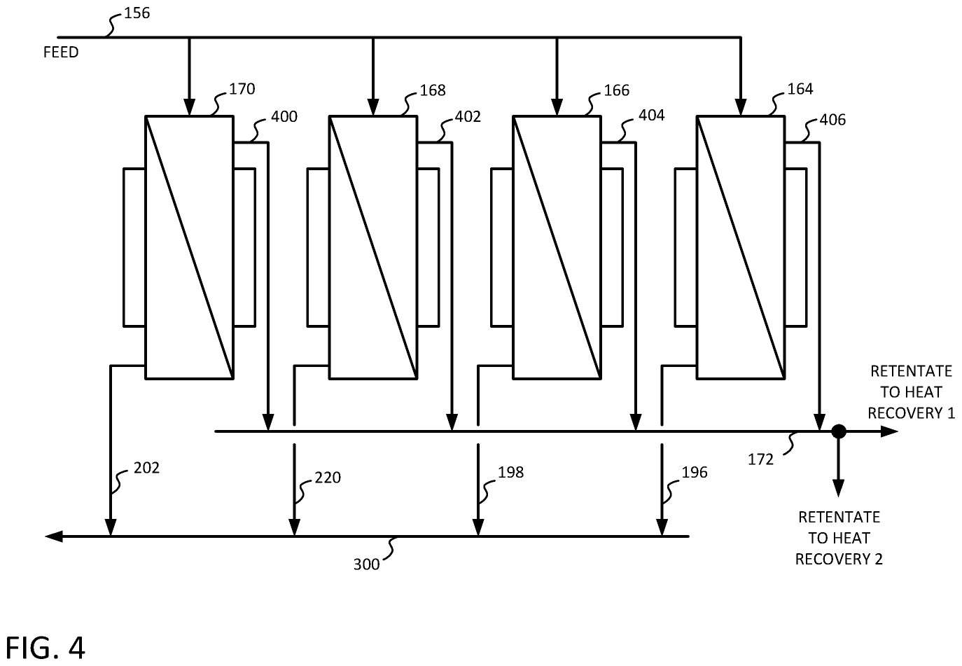

FIG. 4 illustrates an example layout in which the membranes provide permeate stream outputs to a single permeate header 300 provided to a single downstream process (e.g., a permeate condenser), while providing the retentate stream output to a single retentate header 172 provided to multiple downstream processes (e.g., to first and second retentate heat recovery systems).

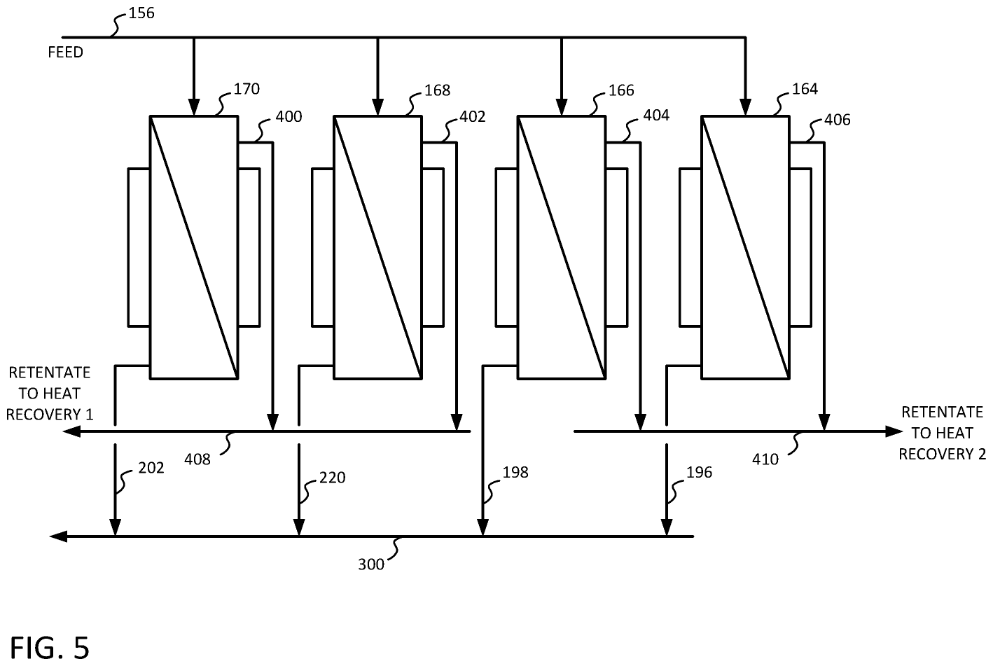

FIG. 5 illustrates an example layout in which the membranes provide permeate stream outputs to a single permeate header 300 provided to a single downstream process (e.g., a permeate condenser), while providing the retentate stream output to multiple retentate headers provided to different downstream processes (e.g., to first and second retentate heat recovery systems).

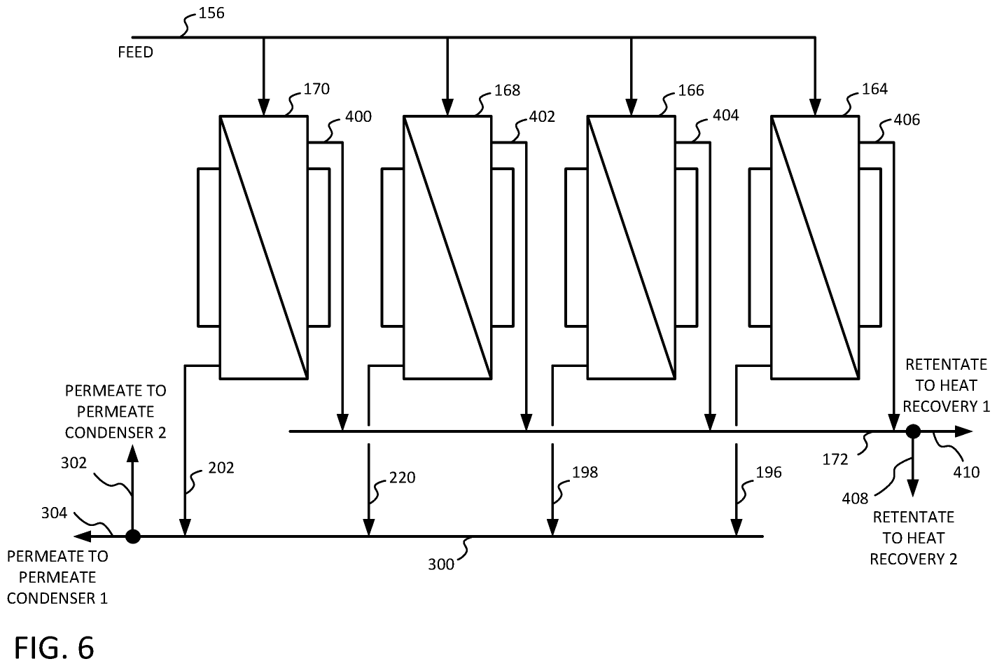

FIG. 6 illustrates an example layout in which the membranes provide permeate stream outputs to a single permeate header provided to multiple downstream process (e.g., a first and a second permeate condenser), while providing the retentate stream output to a single retentate headers provided to multiple downstream processes (e.g., to first and second retentate heat recovery systems).

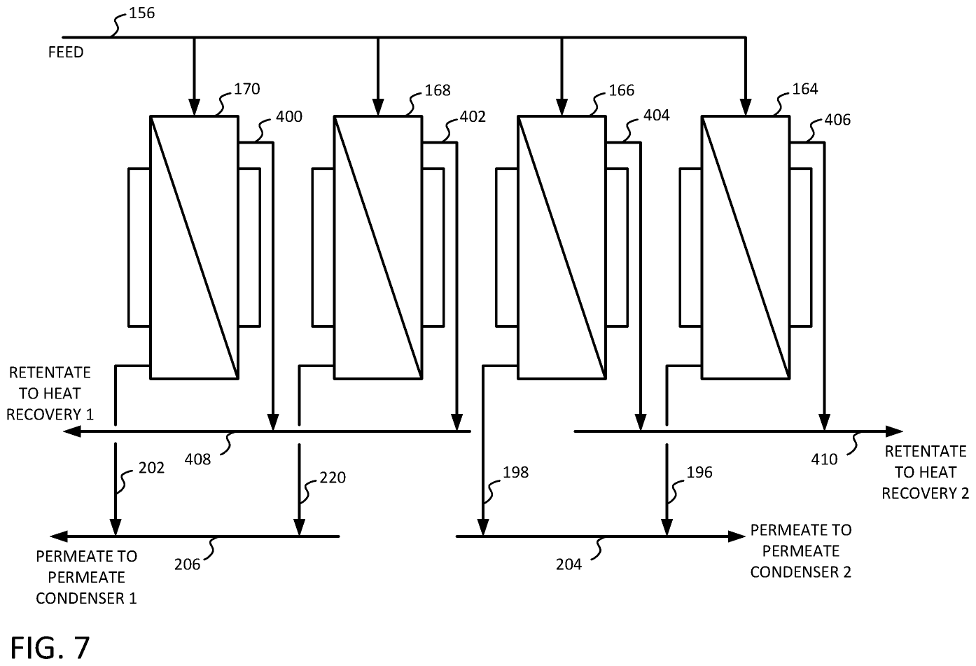

FIG. 7 illustrates an example layout in which the membranes provide permeate stream outputs to multiple permeate headers provided to different downstream process (e.g., a first and a second permeate condenser), while providing the retentate stream output to multiple retentate headers provided to different downstream processes (e.g., to first and second retentate heat recovery systems).

For example, in FIG. 2, the permeate vapor 196 generated by the membrane 164 and the permeate vapor 198 generated by the membrane 166 are each directed to a second permeate header 204. The second permeate header 204 then directs the permeate vapor 196 and 198 to a condenser 136 as a permeate vapor stream to a second permeate header 204. In this example, the permeate vapor stream 208 carried by the second permeate header 204 exchanges heat with a portion of the overhead stream 118 formed by the rectifier/stripper column 112 (via the stream 132) in the condenser 136. Stated differently, the permeate vapor stream 208 carried by second permeate header 204 heats the stream 132 via the condenser 136, thereby cooling the permeate vapor stream to a liquid. Subsequent to exchanging heat in the condenser 136, the permeate liquid stream 212 is directed to the distillation column 154. In other examples, the permeate vapor stream 208 may be used to heat another suitable process stream within the organic solvent production system 100 (e.g., any suitable cold stream such as a 190P stream, scrubber bottoms, cook water, regen, cooled permeate, beer, boiler feed water, fresh make-up water, a separation system feed, etc.).

Differently than the permeate vapor 196 and 198, the permeate vapor 220 generated by the membrane 168 and the permeate vapor 202 generated by the membrane 170 are each directed to a first permeate header 206 in this example. The first permeate header 206 then directs the permeate vapor 220 and 202 to a condenser 216 via the permeate vapor stream 210. The condenser 216 thereby condenses the permeate vapor stream 210 to a liquid. The permeate liquid stream 218 may thereafter be directed to a heat exchanger 192. In some aspects, the permeate liquid stream 218 may be heated by the retentate liquid stream 134 within the heat exchanger 192. The heated permeate liquid stream 214 may then be directed to the distillation column 154.

In some examples, the organic solvent production system 100 may only include one of the second permeate header 204 and the first permeate header 206. For instance, the organic solvent production system 100 may only include the membranes 164, 166, and 168. As such, the permeate vapor 196 and the permeate vapor 198 may be directed to the permeate header 204 and thereafter to the condenser 136, whereas the permeate vapor 220 may be directed straight to the condenser 216. In other examples, the organic solvent production system 100 may include three or more permeate headers that are each in fluid communication with a separate condenser. In some examples, three or more membranes may each be in fluid communication with a single permeate header such that the each direct their respective generated permeate vapor to the single permeate header.

In another example shown in FIG. 3, the permeate vapor 196, 198, 220, and 202 generated by each of the membranes 164, 166, 168, and 170 is all directed to the permeate header 300. In this example, the permeate header 300 then splits into a permeate vapor stream 302 and a permeate vapor stream 304. In other examples, the permeate header 300 may split into three or more streams. The permeate vapor stream 302 is directed to a first condenser (e.g., the condenser 136) whereas the permeate vapor stream 304 is directed to a second condenser (e.g., the condenser 216). The permeate vapor streams 302 and 304 may each exchange heat with another process stream within the respective condensers.

The amount of permeate vapor in each of the permeate vapor streams 302 and 304 may be different and can be controlled. For example, the organic solvent production system 100 may include a control valve on each of the piping through which the permeate vapor streams 302 and 304 flow. Each control valve is adjustable to partially or fully restrict flow through its respective piping, which thereby controls the amount of permeate vapor in each of the permeate vapor streams 302 and 304. In some aspects, a control valve may be adjusted manually. In other aspects, a control valve may be adjusted automatically via a controller having a processor in communication with a memory, the processor being in communication with each of the control valves. In various examples, the control valves may be adjusted based on the process conditions (e.g., flow, pressure, temperature, etc.) of each of the process streams against which the respective latent energy of the permeate vapor streams 302 and 304 is being recovered. In such examples, the control valves may be adjusted so that the permeate vapor in each of the permeate vapor streams 302 and 304 is fully condensed against the respective process streams.

As such, according to one or more of the examples, the organic solvent production system 100 spreads the permeate vapor 196, 198, 220, and 202 generated by the set of membranes 162 out to various numbers of condensers. The duty on each condenser is thereby reduced as compared to a condenser that receives all of the permeate vapor 196, 198, 220, and 202 generated by the set of membranes 162. In this way, the organic solvent production system 100 enables recovering the heat (e.g., energy) of the permeate vapor 196, 198, 220, and 202 against a larger array of process streams than typical systems.

In some aspects, the retentate generated by the set of membranes 162 may be split into at least two different streams, such as into two or more retentate headers. Splitting the generated retentate enables producing two retentate streams of different organic solvent purities (e.g., fuel grade and pharmaceutical grade) for different applications at the same time. As will be appreciated, different grades of purity may be specified by different jurisdictions for different use cases, and the described system is capable of being configured to simultaneously produce two or more grades having different purity levels of the solvent, which may match corresponding definitions set in different jurisdictions.

FIG. 4 illustrates an example in which the retentate vapor 400 generated by the membrane 170 and the retentate vapor 402 generated by the membrane 168 are each directed to a retentate header 408, whereas the retentate vapor 404 generated by the membrane 166 and the retentate vapor 406 generated by the membrane 164 are each directed to a retentate header 410. The retentate vapor 400 and 402 directed into the retentate header 408 may be directed to a first retentate condenser and thereafter to a first storage tank. The retentate vapor 404 and 406 directed into the retentate header 410 may be directed to a second retentate condenser and thereafter to a second storage tank. The retentate vapor from a first and second header can be sent to two separate evaporators. Also, when the retentate vapor is split by two or more headers to produce multiple separate product streams with different purities, retentate vapor from one of the headers can go to the evaporators. Where MSUs are operating (as shown in FIG. 1) retentate vapor from one of the headers can be directed to the MSU 200P vapor if the quality of this stream is the same as the MSU 200P vapor. The retentate vapor from the other header can go elsewhere to recover latent energy available in the steam such as against a 190P stream, a regen stream, a beer feed stream, reboiling distillation column bottoms stream, a system feed stream, a cook water stream, etc. In some aspects, the retentate vapor may be recovered against the aforementioned stream in separate condensers in addition to or instead of via evaporators.

As illustrated in FIG. 6, the permeate vapor 196, 198, 220, and 202 generated by each of the membranes 164, 166, 168, and 170 is all directed to the permeate header 300. As described in relation to FIG. 3, the permeate header 300 may split into two or more streams (e.g., streams 302, 304) in various embodiments so that the permeate vapor can be directed to one or more downstream systems (e.g., a first condenser and a second condenser) and exchange heat with other process steams in the respective systems. In addition or alternatively to splitting the permeate header 300, the example illustrated in FIG. 6 shows that the vapor in the retentate vapor carried by retentate header 172 may also be split into a first retentate stream and a second retentate stream via respective retentate headers, which can be directed to one or more downstream systems (e.g., an evaporator and a condenser or separate condensers) and exchange heat with other process steams in the respective systems. Although illustrated as a two-way split, in other examples, the retentate vapor stream carried by the retentate header 172 may split into three or more streams.

The amount of retentate vapor in each of the retentate vapor streams carried by the respective retentate headers may be different and can be controlled. For example, the organic solvent production system 100 may include a control valve on each of the piping through which the retentate vapor streams flow. Each control valve is adjustable to partially or fully restrict flow through its respective piping, which thereby controls the amount of retentate vapor in each of the retentate vapor headers 408 and 410. In some aspects, a control valve may be adjusted manually. In other aspects, a control valve may be adjusted automatically via a controller having a processor in communication with a memory, the processor being in communication with each of the control valves. In various examples, the control valves may be adjusted based on the process conditions (e.g., flow, pressure, temperature, etc.) of each of the process streams against which the respective latent energy of the retentate vapor streams carried by the retentate headers 408 and 410 is being recovered. In such examples, the control valves may be adjusted so that the retentate vapor in each of the retentate headers 408 and 410 is fully condensed against the respective process streams.

As such, the organic solvent production system 100 spreads the retentate vapor generated by the set of membranes 162 out to two or more condensers. The duty on each condenser is thereby reduced as compared to a condenser that receives all of the retentate vapor generated by the set of membranes 162. In this way, the organic solvent production system 100 enables recovering the heat (e.g., energy) of the retentate vapor against a larger array of process streams than typical systems.

As noted above, the retentate vapor in the second retentate header 408 may have a different organic solvent purity than the retentate vapor in the first retentate header 410. A contributing factor in enabling the difference in organic solvent purities is the membranes 168 and 170 (which direct their retentate vapor into the retentate header 408) may be operated at a different vacuum pressure than the membranes 164 and 166 (which direct the retentate vapor into the retentate header 410). For example, the membranes 168 and 170 may be operated at a deeper vacuum pressure than the membranes 164 and 166. The deeper vacuum pressure of the membranes 168 and 170 contributes to the retentate vapor 402 and 400 generated by the membranes 168 and 170 being of a higher organic solvent purity (e.g., pharmaceutical grade) than the purity (e.g., fuel grade) of the retentate vapor 406 and 404 generated by the membranes 164 and 166 operating at a lower vacuum pressure.

It will be appreciated that other design considerations known in the art contribute to the difference in organic solvent purities of the retentate generated by the membranes 168 and 170 as compared to the retentate generated by the membranes 164 and 166. For example, the membranes 168 and 170 may have a greater membrane surface area than the membranes 164 and 166.

In some examples, the organic solvent production system 100 may only include one of the retentate header 408 and the retentate header 410. For instance, the organic solvent production system 100 may only include the membranes 164, 166, and 168. As such, the retentate vapor 406 and the retentate vapor 404 may be directed to the retentate header 410 and thereafter to a first retentate condenser, whereas the retentate vapor 402 may be directed straight to a second retentate condenser. In other examples, the organic solvent production system 100 may include three or more retentate headers that are each in fluid communication with a separate retentate condenser. In some examples, three or more membranes may each be in fluid communication with a single retentate header such that the each direct their respective generated retentate vapor to the single retentate header.

In the example of FIG. 4, the permeate vapor 196, 198, 220, 202 generated by the membranes 164, 166, 168, 170 is all directed into a single permeate header 300. Stated differently, in some aspects, the organic solvent production system 100 may include one or more retentate headers without including any permeate headers. In other examples, the permeate vapor 196, 198, 220, 202 generated by the membranes 164, 166, 168, 170 in FIG. 4 may be split into two or more streams, such as that described for FIGS. 1 to 3. Stated differently, in such other examples the organic solvent production system 100 may include both permeate headers and retentate headers.

It will be appreciated by one having skill in the pertinent art that many other suitable organic solvent production systems beyond the organic solvent production system 100 may be constructed embodying the permeate heat recovery concepts and/or the retentate flexibility concepts described herein, and that the organic solvent production system 100 is merely one example. For example, the organic solvent production system 100 may include two separate distillation columns that each receive a portion of the feed stream 102. In another example, the organic solvent production system 100 may include a rectifier/stripper column 112 in fluid communication with a separate side stripper column. In another example, the organic solvent production system 100 might not include a molecular sieve unit 142. Additionally, in various examples, the intermediate components (e.g., reboilers, condensers, economizers, vaporizers, storage tanks, flash vessels, etc.) of the organic solvent production system 100 may be rearranged, some may be removed, or new suitable ones may be added.

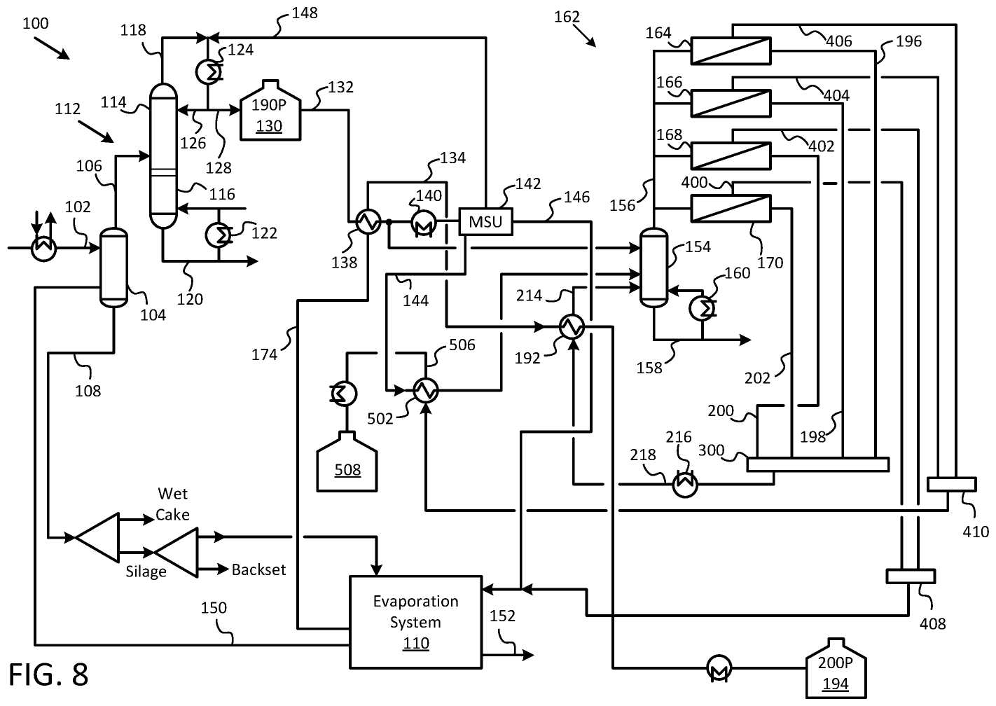

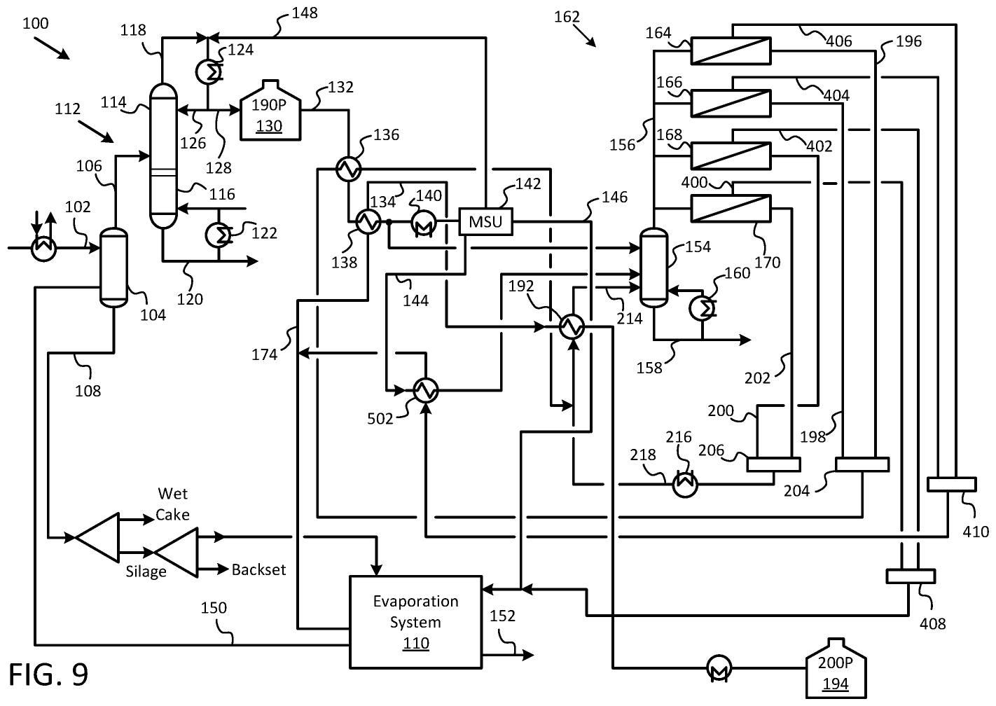

FIG. 8 illustrates an example organic solvent production system 100 including split retentate headers, according to an aspect of the present disclosure. Similarly to the production system 100 illustrated in FIG. 1, the example organic solvent production system 100 can be described as including four subsystems: (i) a feed stripping subsystem, (ii) a rectifying distillation subsystem, (iii) a dehydration subsystem, and (iv) an evaporation subsystem. However, in contrast to the system 100 of FIG. 1, the system 100 illustrated in FIG. 8 uses a split retentate header arrangement rather than a split permeate header arrangement. FIG. 9 provides additional detail showing that the routing of FIG. 1 may be freely combined with the routing illustrated in FIG. 8 to provide a system using both a split permeate header arrangement and a split retentate header arrangement. Accordingly, discussion of FIG. 5 focuses on the differences between FIG. 8 and FIG. 1 in relation to the routing for the outputs of the set of membranes 162.

The output permeate vapor streams 196, 198, 220, 202 from the individual membranes 164, 166, 168, 170 are routed via a single permeate header 300 to a permeate condenser 216, which produces a permeate liquid stream 218 that is routed to a permeate pre-heater 192 before being routed as a heated permeate liquid stream 214 to the distillation column 154 once pre-heated by the 200P liquid stream 134 routed to the permeate pre-heater 192.

Each of the first retentate header 408 and the second retentate header 410 may be routed to different components of the system 100. For example, in FIG. 8, the first retentate header 408 routes a first portion of the retentate vapor to the evaporation system 110, while the second retentate header 410 routes a second portion of the retentate vapor to a retentate condenser 502. The retentate condenser 502, in turn, routes a first portion of the condensed MSU Regen to a distillation column 154 and a second portion of the condensed retentate 506 to one or more retentate storage tanks 508. In various embodiments, the routing of the retentate vapor from the individual membranes 164, 166, 168, 170 may be different from that illustrated in FIG. 8 (e.g., the retentate vapor from membranes 164, 166, 168 may be provided as the first retentate header 410 and the retentate vapor from membrane 170 as the second retentate header 408) and may include more or fewer membranes in the set of membranes 162 than are illustrated. Additionally, although illustrated with a split arrangement dividing the output from the set of membranes 162 into a first retentate header 410 and a second retentate header 408, in various embodiments the retentate vapor stream may be divided into three or more portions with various inputs from the set of membranes 162.

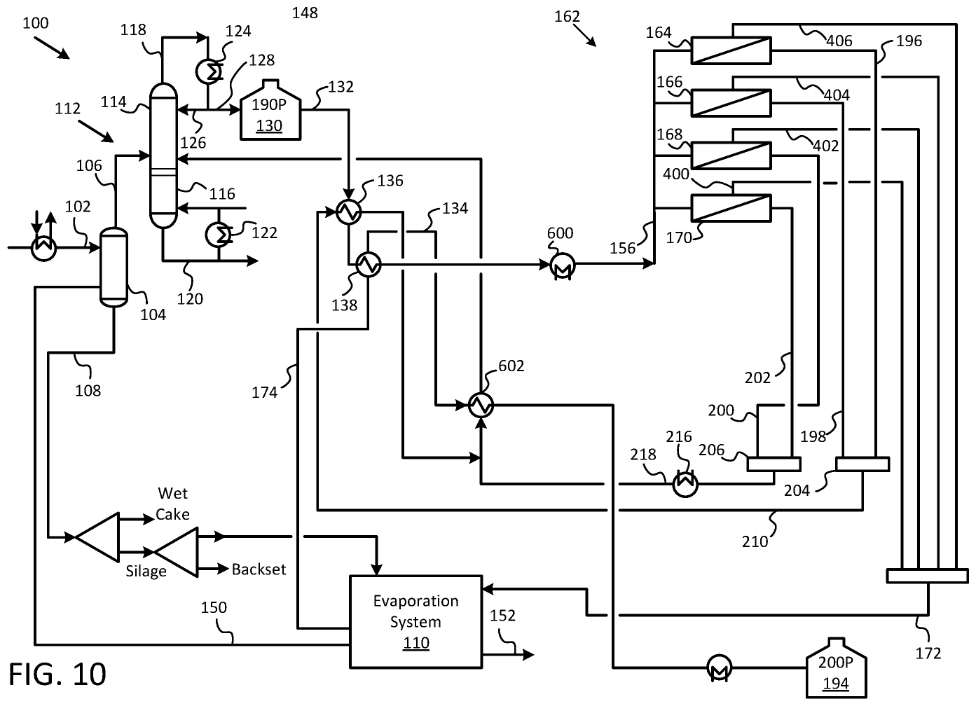

FIG. 10 illustrates an example organic solvent production system including a vaporizer 600, according to an aspect of the present disclosure. Although sharing various similarities with the system shown in FIG. 1, FIG. 10 show that the split header design can also be applied to an organic solvent production system where the MSUs have been replaced completely with membranes. In FIG. 10, the MSU are omitted and the design includes vaporizers 600 and membranes only. Latent energy from the permeate stream from the split headers is recovered against suitable streams (e.g., Cook Water and 190P in the illustrated example, but other cold streams are contemplated). Condensed permeate is then pre-heated via a permeate preheater 602 using thermal energy from the retentate stream before the condensed permeate is sent back to the Rectifier-Side Stripper Column 114/116 for reprocessing.

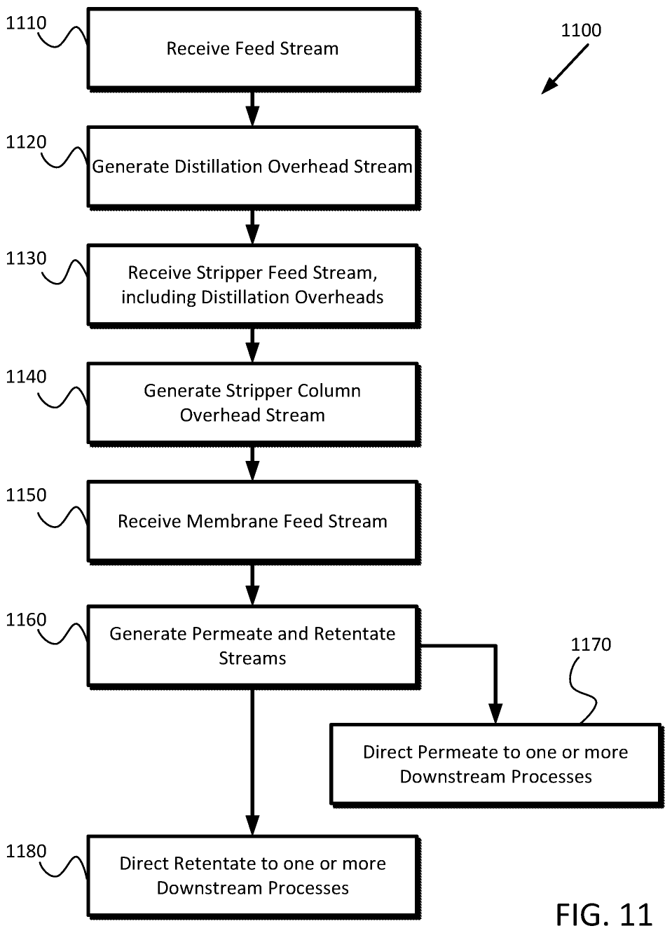

FIG. 11 is a flowchart of an example method 1100 for operating a dehydration system with a split header design, according to an aspect of the present disclosure. Method 1100 begins at block 1110 where a distillation column receives a feed stream with a solution including a solvent at a first purity level.

At block 1120, the distillation column generates a distillation overhead stream from the feed stream, having a second purity level higher than the first purity level for the solvent.

At block 1130, a stripper column receives a stripper feed stream, which includes at least a portion of the distillation column overhead stream (generated per block 1120).

At block 1140, the stripper column generates a stripper column overhead stream from the stripper feed stream.

At block 1150, a set of membranes receive a membrane feed stream, which may include at least a portion of the stripper column overhead stream (generated per block 1140). In various embodiments, blocks 1110-1140 may be replaced by or augmented by various other operations to generate different or additional inputs to the membrane feed streams or to generate a membrane feed stream by other methodologies. In various aspects, the set of membranes includes at least three membranes. Each membrane of the set of membranes receiving a respective portion of the membrane feed stream, but an operator may cycle which membranes actively receive a feed stream at any given time (e.g., to rejuvenate, repair, or replace a membrane; to control a variable input flowrate to produce a consistent through-flow rate in the active membranes; to provide different purity levels of the solvent in the output streams; etc.).

At block 1160, the (active) membranes of the set of membranes each generate a respective permeate stream and a respective retentate stream from the respective portion of the membrane feed stream. The total permeate stream of the system is made up of the combined total of the individual respective permeate streams from each active membrane and the total retentate stream of the system is made up of the combined total of the individual respective retentate streams from each active membrane.

At block 1170, the system directs the permeate to one or more downstream processes. In some aspects, the total permeate is received by a single header connected to each active membrane, and is directed to a single downstream process. In some aspects, the total permeate is received by a single header connected to each active membrane, and is directed to a two or more downstream processes. In some aspects, a first non-zero portion of the total permeate is received by a first header connected to a first subset of the active membranes, and is directed to a first downstream process, and a second non-zero portion of the total permeate is received by a second header connected to a second subset of the active membranes, and is directed to a second downstream process.

At block 1180, the system directs the retentate to one or more downstream processes. In some aspects, the total retentate is received by a single header connected to each active membrane, and is directed to a single downstream process. In some aspects, the total retentate is received by a single header connected to each active membrane, and is directed to a two or more downstream processes. In some aspects, a first non-zero portion of the total retentate is received by a first header connected to a first subset of the active membranes, and is directed to a first downstream process, and a second non-zero portion of the total retentate is received by a second header connected to a second subset of the active membranes, and is directed to a second downstream process.

Block 1170 and block 1180 may be performed in parallel according to the different layouts, as is detailed in Table 1.

TABLE 1
Membrane Output Routing

| Permeate | Retentate |
|---|---|
| Single header with single downstream process | Single header with single downstream process |
| Single header split to multiple downstream | Single header with single downstream process |
| processes | |
| Multiple headers associated with multiple | Single header with single downstream process |
| downstream processes | |
| Single header with single downstream process | Single header split to multiple downstream |
| | processes |
| Single header split to multiple downstream | Single header split to multiple downstream |
| processes | processes |
| Multiple headers associated with multiple | Single header split to multiple downstream |
| downstream processes | processes |
| Single header with single downstream process | Multiple headers associated with multiple |
| | downstream processes |
| Single header split to multiple downstream | Multiple headers associated with multiple |
| processes | downstream processes |
| Multiple headers associated with multiple | Multiple headers associated with multiple |
| downstream processes | downstream processes |

The various downstream processes may include condensing operations and/or heat exchanging operations, where retained heat in the retentate and/or permeate is exchanged against a colder stream before that stream is provided to another section of the solvent production plant; thereby reducing energy expenditure to pre-heat various streams or cool the retentate or permeate before storing for delivery as a product.

Certain terms are used throughout the description and claims to refer to particular features or components. As one skilled in the art will appreciate, different persons may refer to the same feature or component by different names. This document does not intend to distinguish between components or features that differ in name but not function.

As used herein, various chemical compounds are referred to by associated element abbreviations set by the International Union of Pure and Applied Chemistry (IUPAC), which one of ordinary skill in the relevant art will be familiar with. Similarly, various units of measure may be used herein, which are referred to by associated short forms as set by the International System of Units (SI), which one of ordinary skill in the relevant art will be familiar with.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

The invention is claimed as follows:

1. An organic solvent production system comprising:
a distillation column configured to receive a feed stream and generate a distillation column overhead stream from the feed stream;
a stripper column arranged to indirectly receive a stripper feed stream including at least a portion of the distillation column overhead stream, the stripper column configured to generate a stripper column overhead stream from the stripper feed stream;
at least three membranes arranged to each receive a respective portion of a membrane feed stream including at least a portion of the stripper column overhead stream, wherein each of the at least three membranes is configured to generate from its respective portion of the membrane feed stream a respective permeate and a respective retentate, the respective permeates together composing a total permeate and the respective retentates together composing a total retentate;
a first condenser; and
a second condenser,
wherein each of the at least three membranes is in fluid communication with at least one of the first condenser and the second condenser such that a first non-zero portion of the total permeate generated by the at least three membranes is directed to the first condenser and a second non-zero portion of the total permeate generated by the at least three membranes is directed to the second condenser.

2. The system of claim 1, wherein a first membrane and a second membrane of the at least three membranes are each in fluid communication with a first permeate header such that the respective permeates of the first and second membranes are both directed to the first permeate header, and wherein the first permeate header is in fluid communication with the first condenser and not with the second condenser such that the first non-zero portion of the total permeate includes the respective permeates generated by the first and second membranes.

3. The system of claim 2, wherein a third membrane of the at least three membranes is in fluid communication with the second condenser and not with the first condenser such that the second non-zero portion of the total permeate includes the permeate generated by the third membrane.

4. The system of claim 2, wherein the at least three membranes includes a third membrane and a fourth membrane, wherein the third and fourth membranes are each in fluid communication with a second permeate header such that the respective permeates of the third and fourth membranes are both directed to the second permeate header, and wherein the second permeate header is in fluid communication with the second condenser and not with the first condenser such that the second non-zero portion of the total permeate includes the respective permeates generated by the third and fourth membranes.

5. The system of claim 1, further comprising a first valve and a second valve, the first valve configured and arranged to adjust a flow of the first non-zero portion of the total permeate, and the second valve configured and arranged to adjust a flow of the second non-zero portion of the total permeate.

6. The system of claim 5, wherein the first and second valves are arranged such that adjustment of at least one of the first valve and the second valve adjusts an amount of the first portion of the total permeate as compared to the second portion.

7. The system of claim 1, further comprising a first process stream directed to the first condenser, wherein the first non-zero portion of the total permeate exchanges energy with the first process stream in the first condenser.

8. The system of claim 7, further comprising a second process stream directed to the second condenser, wherein the second non-zero portion of the total permeate exchanges energy with the second process stream in the second condenser.

9. The system of claim 1, wherein the organic solvent is one in the group consisting of ethanol, iso-butanol, and isopropanol.

10. The system of claim 1, wherein the organic solvent is ethanol.

11. The system of claim 1, wherein each of the at least three membranes is in fluid communication with an evaporation system, the evaporation system including a plurality of evaporators, such that the respective retentates generated by the at least three membranes are directed to at least one evaporator of the plurality of evaporators.

12. The system of claim 1, further comprising a stripper/rectifier column arranged to receive the distillation column overhead stream as a vapor, the stripper/rectifier column configured to generate a stripper/rectifier column overhead stream from the vaporous distillation column overhead stream, wherein the stripper column is arranged to receive at least a portion of the stripper/rectifier column overhead stream, and wherein the stripper/rectifier column overhead stream includes the at least a portion of the distillation column overhead stream.

13. The system of claim 1, further comprising a molecular sieve unit arranged to receive a molecular sieve unit feed stream including at least a portion of the distillation column overhead stream, the molecular sieve unit configured to generate a regenerate stream from the molecular sieve unit feed stream, wherein the stripper column is arranged to directly receive the regenerate stream, and wherein the regenerate stream includes the at least a portion of the distillation column overhead stream.

14. The system of claim 1, wherein each of the at least three membranes is in fluid communication with at least one of a third condenser and a fourth condenser such that a first non-zero portion of the total retentate generated by the at least three membranes is directed to the third condenser and a second non-zero portion of the total retentate generated by the at least three membranes is directed to the fourth condenser.

15. The system of claim 1, further comprising:
a third condenser; and
an evaporator,
wherein each of the at least three membranes is in fluid communication with at least one of the third condenser and the evaporator such that a first non-zero portion of the total retentate generated by the at least three membranes is directed to the third condenser and a second non-zero portion of the total retentate generated by the at least three membranes is directed to the evaporator.

16. An organic solvent production method, comprising:
receiving a feed stream at a distillation column;
generating a distillation column overhead stream from the feed stream via the distillation column;
receiving a stripper feed stream at a stripper column, the stripper feed stream including at least a portion of the distillation column overhead stream;
generating a stripper column overhead stream from the stripper feed stream via the stripper column;
receiving a respective portion of a membrane feed stream at each of at least three membranes, the membrane feed stream including at least a portion of the stripper column overhead stream;
generating a respective permeate and a respective retentate from the respective portion of the membrane feed stream at each respective membrane of the at least three membranes, the respective permeates together composing a total permeate and the respective retentates together composing a total retentate;
directing a first non-zero portion of the total permeate to a first condenser; and
directing a second non-zero portion of the total permeate to a second condenser.

17. The organic solvent production method of claim 16, exchanging energy of the first non-zero portion of the total permeate with a first process stream in the first condenser.

18. The organic solvent production method of claim 17, wherein the first process stream is one of the streams in the group consisting of: a 190P stream, a scrubber bottoms stream, a cook water stream, a regen stream.

19. The organic solvent production method of claim 17, further comprising exchanging energy of the second non-zero portion of the total permeate with a second process stream in the second condenser.

20. The organic solvent production method of claim 16, wherein the first non-zero portion of the total permeate includes all of the respective permeates generated by each of a first membrane and a second membrane of the at least three membranes, and wherein the second non-zero portion of the total permeate includes all of the permeate generated by a third membrane of the at least three membranes.

21. The organic solvent production method of claim 20, wherein the second non-zero portion of the total permeate includes all of the respective permeates generated by each of the third membrane and a fourth membrane of the at least three membranes.

22. The organic solvent production method of claim 16, further comprising:
directing a first non-zero portion of the total retentate to a third condenser; and
directing a second non-zero portion of the total retentate to an evaporator.

23. An organic solvent production system comprising:
a distillation column configured to receive a feed stream and generate a distillation column overhead stream from the feed stream;
a stripper column arranged to indirectly receive a stripper feed stream including at least a portion of the distillation column overhead stream, the stripper column configured to generate a stripper column overhead stream from the stripper feed stream;
at least three membranes arranged to each receive a respective portion of a membrane feed stream including at least a portion of the stripper column overhead stream, wherein each of the at least three membranes is configured to generate from its respective portion of the membrane feed stream a respective permeate and a respective retentate, the respective permeates together composing a total permeate and the respective retentates together composing a total retentate;
a condenser; and
an evaporator,
wherein each of the at least three membranes is in fluid communication with at least one of the condenser and the evaporator such that a first non-zero portion of the total retentate generated by the at least three membranes is directed to the condenser and a second non-zero portion of the total retentate generated by the at least three membranes is directed to the evaporator.

24. The organic solvent production system of claim 23, further comprising:
a second condenser; and
a third condenser,
wherein each of the at least three membranes is in fluid communication with at least one of the second condenser and the third condenser such that a first non-zero portion of the total permeate generated by the at least three membranes is directed to the second condenser and a second non-zero portion of the total permeate generated by the at least three membranes is directed to the third condenser.

25. An organic solvent production method comprising:
receiving a feed stream at a distillation column;
generating a distillation column overhead stream from the feed stream via the distillation column;
receiving a stripper feed stream at a stripper column, the stripper feed stream including at least a portion of the distillation column overhead stream;
generating a stripper column overhead stream from the stripper feed stream via the stripper column;
receiving a respective portion of a membrane feed stream at each of at least three membranes, the membrane feed stream including at least a portion of the stripper column overhead stream;
generating a respective permeate and a respective retentate from the respective portion of the membrane feed stream at each respective membrane of the at least three membranes, the respective permeates together composing a total permeate and the respective retentates together composing a total retentate;
directing a first non-zero portion of the total retentate to a condenser; and
directing a second non-zero portion of the total retentate to an evaporator.

26. The organic solvent production method of claim 25, further comprising:
directing a first non-zero portion of the total permeate to a second condenser; and
directing a second non-zero portion of the total permeate to a third condenser.
